(19) United States
(12) Patent Application Publication
(10) Pub. No.: US 2023/0233470 A1
(43) Pub. Date: Jul. 27, 2023

(54) Formulations

(71) Applicant: ViiV Healthcare Company, Wilmington, DE (US)

(72) Inventors: Ian Paul CONN, Ware, Hertfordshire (GB); Mark Robert DAVIES, Ware, Hertfordshire (GB); Jose Maria FUEYO, Ware, Hertfordshire (GB); Joanne HEAFIELD, Ware, Hertfordshire (GB); Trevor Martin SHREEVES, Ware, Hertfordshire (GB)

(73) Assignee: ViiV Healthcare Company, Wilmington, DE (US)

(21) Appl. No.: 18/001,634

(22) PCT Filed: Jun. 23, 2021
(86) PCT No.: PCT/IB2021/055535

§ 371 Date: Dec. 13, 2022

(30) Foreign Application Priority Data
Jun. 25, 2020 (GB) 2009685.5

(51) Int. Cl. A61K 9/24; A61K 9/20; A61K 31/513; A61K 31/52

(52) U.S. Cl. CPC A61K 9/209; A61K 9/2018; A61K 9/2095; A61K 31/513; A61K 31/52

(57) ABSTRACT

The present invention relates to formulations comprising dolutegravir or a pharmaceutically acceptable salt thereof, abacavir or a pharmaceutically acceptable salt thereof and lamivudine, processes for making such formulations, and the use of such formulations in the treatment of HIV infection, in particular in the treatment of HIV infection in pediatric patients.

### FIELD OF THE INVENTION

The present invention relates to formulations comprising dolutegravir or a pharmaceutically acceptable salt thereof, abacavir or a pharmaceutically acceptable salt thereof and lamivudine, processes for making such formulations, and the use of such formulations in the treatment of HIV infection, in particular in the treatment of HIV infection in pediatric patients.

### BACKGROUND TO THE INVENTION

Human immunodeficiency virus (“HIV”) infection and related diseases are a major public health problem worldwide. Human immunodeficiency virus type 1 (“HIV-1”) is a retrovirus which encodes three enzymes that are required for viral replication: reverse transcriptase, protease, and integrase. Drugs targeting reverse transcriptase, protease and integrase are in wide use and have shown effectiveness, particularly when employed in combination.

HIV infection however remains a major medical problem, with tens of millions of people still infected worldwide. The World Health Organization reported that 2.6 million children of less than 15 years of age were living with HIV-1 in 2014. Although therapeutic options for this patient population have improved, additional pediatric formulations of antiretroviral agents are needed.

Dolutegravir is an integrase strand transfer inhibitor (INSTI). Dolutegravir inhibits HIV integrase by binding to the integrase active site and blocking the strand transfer step of retroviral DNA integration which is essential for the HIV replication cycle.

The chemical name of dolutegravir is (4R,12aS)-N-[(2,4-difluorophenyl)methyl]-7-hydroxy-4-methyl-6,8-dioxo-3,4,12,12a-tetrahydro-2H-pyrido[5,6]pyrazino[2,6-b][1,3]oxazine-9-carboxamide (CAS Registry Number 1051375-16-6). Dolutegravir has the following structural formula:

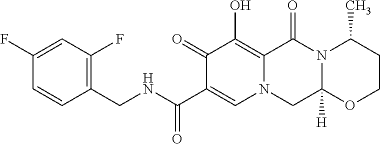 [SMILES: C[C@@H]1CCO[C@H]2Cn3cc(C(=O)NCc4ccc(F)cc4F)c(=O)c(O)c3C(=O)N12]

Dolutegravir sodium (TIVICAY) is approved for use in a broad population of HIV-infected patients. It is recommended for first-line treatment of HIV-1 infected adults due to it potency, high barrier to resistance and tolerability. It is also approved for children aged 6 to 18 years of age.

Abacavir is a nucleoside reverse transcriptase inhibitor (NRTI). Abacavir hemisulfate (empirical formula C₁₄H₁₈N₆O.0.5 H₂SO₄) is approved for use in the treatment of HIV-infected patients in combination with other HIV medicines.

The chemical name of abacavir hemisulfate is (1S,4R-4-[2-amino-6-cyclopropylamino)-9H-purin-9-yl]-2-cyclopentene-1-methanol hemisulfate (CAS Registry Number 188062-50-2). Abacavir hemisulfate has the following structural formula:

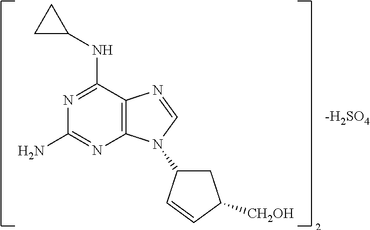

Lamivudine is also a NRTI which is approved for use in the treatment of HIV-infected patients.

The chemical name of lamivudine is 2′,3′-dideoxy-3′-thiacytidine (CAS Registry Number 134678-17-4). Lamivudine has the following structural formula:

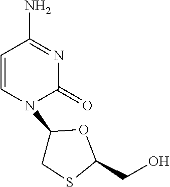 [SMILES: Nc1ccn([C@@H]2CS[C@H](CO)O2)c(=O)n1]

TRIUMEQ® is a fixed dose combination of dolutegravir sodium, abacavir hemisulfate and lamivudine which is approved for the treatment of HIV-infected patients. The adult dosage form of TRIUMEQ® is a tablet comprising 50 mg (free acid equivalent) of dolutegravir sodium, 600 mg (free base equivalent) of abacavir hemisulfate and 300 mg of lamivudine. However, some patients, in particular pediatric patients, having difficulty swallowing tablets and generally require a different oral drug delivery system. There remains a need for alternative formulations of dolutegravir or a pharmaceutically acceptable salt thereof, abacavir or a pharmaceutically acceptable salt thereof and lamivudine for use in the treatment of HIV infection in certain patients, in particular pediatric patients.

### SUMMARY OF THE INVENTION

In a first aspect, the present invention provides a dispersible tablet formulation comprising dolutegravir or a pharmaceutically acceptable salt thereof, abacavir or a pharmaceutically acceptable salt thereof, lamivudine and at least one taste masking agent.

In a second aspect, the present invention provides a process for making a dispersible tablet formulation comprising dolutegravir or a pharmaceutically acceptable salt thereof, abacavir or a pharmaceutically acceptable salt thereof, lamivudine and at least one taste masking agent comprising mixing dolutegravir or a pharmaceutically acceptable salt thereof, abacavir or a pharmaceutically acceptable salt thereof, lamivudine and at least one taste masking agent.

In a third aspect, the present invention provides a dispersible tablet formulation comprising dolutegravir or a pharmaceutically acceptable salt thereof, abacavir or a pharmaceutically acceptable salt thereof, lamivudine and at least one taste masking agent for use in therapy.

In a fourth aspect, the present invention provides a dispersible tablet formulation comprising dolutegravir or a pharmaceutically acceptable salt thereof, abacavir or a pharmaceutically acceptable salt thereof, lamivudine and at least one taste masking agent for use in the treatment of HIV infection, in particular HIV infection in a pediatric patient.

In a fifth aspect, the present invention provides a method of treating HIV infection, for example HIV infection in a pediatric patient, which method comprises administering to said patient a dispersible tablet formulation comprising dolutegravir or a pharmaceutically acceptable salt thereof, abacavir or a pharmaceutically acceptable salt thereof, lamivudine and at least one taste masking agent.

In a sixth aspect, the present invention provides a kit comprising a dispersible tablet formulation comprising dolutegravir or a pharmaceutically acceptable salt thereof, abacavir or a pharmaceutically acceptable salt thereof, lamivudine and at least one taste masking agent, together with instructions for the use thereof for the treatment of HIV infection.

In a seventh aspect, the present invention provides a combination of a dispersible tablet formulation comprising dolutegravir or a pharmaceutically acceptable salt thereof, abacavir or a pharmaceutically acceptable salt thereof, lamivudine and at least one taste masking agent, together with another therapeutic agent.

### DESCRIPTION OF DRAWINGS/FIGURES

FIG. 1 is a flow diagram of the manufacturing process for preparing dolutegravir sodium granules following a high shear wet granulation process. The dolutegravir is mixed with intragranular excipients followed by the controlled addition of water to allow the nucleation, a deagglomerating wet milling process, a fluid bed drying process to dryout the granules and a dry comilling step to provide the final granule size product. The granule batch is blended and a portion used to manufacture the tablet formulation.

FIG. 2 is a flow diagram of the manufacturing process for preparing a multilayer tablet formulation. First layer of the bi-layer tablet uses a compression blend obtained by pre-blending lamivudine with sweeteners, filler and disintegrant. This blend is then comilled, followed by an additional blending step with dolutegravir granule (supplied through a wet granulation process) and lubricant. The bilayer tablet is formed with the first layer compression blend and a direct compression second layer that contains abacavir to give a total core weight of 380 mg. Cores are then aqueous film coated.

### DETAILED DESCRIPTION OF THE INVENTION

As used herein, the term “comprise” and variations thereof, such as “comprises” or “comprising”, are to be construed in an open, inclusive sense, that is as “including, but not limited to”.

As used herein, the term “dispersible tablet formulation” refers to a tablet which disperses in aqueous phase, for example water, typically before administration. In one embodiment, the tablet of the invention may disperse after administration direct to mouth. Typically, dispersible tablet formulations are solid pharmaceutical forms which are defined by their rate of disintegration in water and the uniformity of dispersion of the particles into which they disintegrate. Dispersible tablet formulations may also be referred to as “tablets for oral suspension”.

The formulations of the present invention may be used to treat all patients including pediatric patients, adolescent patients and adult patients. In one embodiment, the formulations of the present invention are used to treat pediatric patients. As used herein, the term “pediatric patient” refers to a child of from 0 to 12 years of age, for example 4 weeks to 6 months, 6 months to 2 years or 2 years to 6 years.

As used herein, the term “pharmaceutically acceptable” with respect to a substance refers to that substance which is generally regarded as safe and suitable for use without undue toxicity, irritation, allergic response, and the like, commensurate with a reasonable benefit/risk ratio. “Pharmaceutically acceptable” with regard to excipients includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to a salt of a compound that is pharmaceutically acceptable and that possesses (or can be converted to a form that possesses) the desired pharmacological activity of the parent compound. Such salts include, but are not limited to, acid addition salts formed with inorganic acids such as hydrochloric acid, hydrobromic acid, sulfuric acid, nitric acid, phosphoric acid, and the like; or formed with organic acids such as acetic acid, benzene sulfonic acid, benzoic acid, camphorsulfonic acid, citric acid, ethane sulfonic acid, fumaric acid, glucoheptonic acid, gluconic acid, lactic acid, maleic acid, malonic acid, mandelic acid, methane sulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quatemized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, PA, (2005), at p. 732, Table 38-5, both of which are hereby incorporated by reference herein.

As used herein, the term “salts” includes co-crystals. The term “co-crystal” refers to a crystalline compound comprising two or more molecular components, e.g. wherein proton transfer between the molecular components is partial or incomplete.

As used herein, the term “% w/w” means the weight of a component as a percentage of the total weight of eg the granule or tablet core in which the component is present.

The dispersible tablet formulation according to the invention is stable, can be dispersed in water prior to administration and has improved palatability, and thus increased compliance. In one embodiment, it is believed that it may be possible to administer the dispersible tablet formulation according to the invention direct to mouth.

In a first aspect, the present invention provides a dispersible tablet formulation comprising dolutegravir or a pharmaceutically acceptable salt thereof, abacavir or a pharmaceutically acceptable salt thereof, lamivudine and at least one taste masking agent.

In one embodiment, the dolutegravir or a pharmaceutically acceptable salt thereof is dolutegravir sodium.

In another embodiment, the tablet formulation comprises about 5 mg of dolutegravir free acid equivalent.

In another embodiment, the abacavir or a pharmaceutically acceptable salt thereof is abacavir hemisulfate.

In another embodiment, the tablet formulation comprises about 60 mg of abacavir free base equivalent.

In another embodiment, the tablet formulation comprises about 30 mg of lamivudine.

The taste masking agent is an agent which is able to mask the unpleasant, bitter taste of the active ingredients. Suitable taste masking agents include, but are not limited to, sweeteners and flavouring agents.

In one embodiment, the taste masking agent(s) are present in an amount of about 2% to about 8% w/w in the tablet formulation. In another embodiment, the taste masking agent(s) are present in an amount of about 4% to about 6% w/w in the tablet formulation. In a further embodiment, the taste masking agent(s) are present in an amount of about 5% w/w in the tablet formulation.

In one embodiment, the dispersible tablet formulation of the invention comprises at least one sweetener as the taste masking agent. In a further embodiment, the dispersible tablet formulation of the invention comprises two sweeteners as the taste masking agents.

In one embodiment, the dispersible tablet formulation of the invention comprises two sweeteners as the taste masking agents wherein the first sweetener provides an initial burst of sweetness and the second sweetener provides a more sustained sweetness.

Suitable sweeteners include, but are not limited to, acesulfame potassium (Ace-K), sucralose, sucrose, saccharin and polyols (such as mannitol and sorbitol). For example, in one embodiment, the dispersible tablet formulation of the invention comprises two sweeteners which are acesulfame potassium (Ace-K) and sucralose. Ace-K may give an initial burst of sweetness to mask poor flavour when first taken whereas sucralose may provide a more sustained sweetness to block bitterness.

In one embodiment, the sweetener(s) are present in an amount of about 1% to about 7% w/w in the tablet formulation. In another embodiment, the sweetener(s) are present in an amount of about 3% to about 5% w/w in the tablet formulation. In a further embodiment, the sweetener(s) are present in an amount of about 4% w/w in the tablet formulation.

In another embodiment, the dispersible tablet formulation of the invention comprises two sweeteners and a flavouring agent as the taste masking agents.

Examples of flavouring agents include, but are not limited to, strawberry, orange, banana, raspberry, peach, passion fruit, golden syrup or mixtures thereof. Flavours are readily available from commercial sources such as flavour houses, or can be developed by those skilled in the art. It will be appreciated that preferred flavours assist in masking the taste of the active ingredient(s) of the formulation. In one embodiment, the flavouring agent is strawberry. Representative strawberry flavours may comprise natural flavours, synthetic equivalents thereof, or artificial flavours or mixtures thereof. In another embodiment, the strawberry flavour is strawberry cream flavour, for example PHS-132963 available from the flavour house Givaudan.

In one embodiment, the present invention provides a dispersible tablet formulation comprising dolutegravir or a pharmaceutically acceptable salt thereof, abacavir or a pharmaceutically acceptable salt thereof, lamivudine, Ace-K, sucralose and strawberry cream flavour.

In one embodiment, the flavouring agent is present in an amount of up to about 2% w/w in the tablet formulation. In another embodiment, the flavouring agent is present in an amount of about 0.1% to to about 1% w/w in the tablet formulation. In another embodiment, the flavouring agent is present in an amount of about 0.1% to about 0.2% w/w in the tablet formulation. In a further embodiment, the flavouring agent is present in an amount of about 0.1% w/w in the tablet formulation.

In another embodiment, the dispersible tablet formulation of the invention comprises two sweeteners in an amount of about 3% to about 5% w/w in the tablet formulation and a flavouring agent in an amount of about 0.1% to about 0.2% w/w in the tablet formulation.

Solid state interaction (disproportionation) between dolutegravir sodium and abacavir hemisulfate may occur in the presence of water. Accordingly, in one embodiment, the dispersible tablet formulation is packed with a desiccant, for example a packet of silica gel. Alternatively, in another embodiment, in order to mimimise contact between the active ingredients, the dispersible tablet formulation is a multilayer dispersible tablet formulation. For example, in one embodiment the multilayer dispersable tablet formulation is a bilayer dispersible tablet formulation.

Preferably, the dolutegravir or a pharmaceutically acceptable salt thereof is present within a separate layer of the multilayer dispersible tablet formulation to the abacavir or a pharmaceutically acceptable salt thereof.

In another embodiment, the dolutegravir or a pharmaceutically acceptable salt thereof and lamivudine are present within a separate layer of the multilayer dispersible tablet formulation to the abacavir or a pharmaceutically acceptable salt thereof.

In another embodiment, the dolutegravir or a pharmaceutically acceptable salt thereof and lamivudine layer is in direct contact with the abacavir or a pharmaceutically acceptable salt thereof layer.

Tablet formulations of the invention will typically comprise one or more additional excipients. Excipients should be compatible with the other ingredients of the formulation and physiologically innocuous to the recipient thereof. Examples of suitable excipients are well known to the person skilled in the art of tablet formulation and may be found in, inter alia, “Handbook of Pharmaceutical Excipients”, 7^{th} Ed, 2012. As used herein the term “excipients” is intended to refer to, inter alia, processing aids, basifying agents, solubilisers, glidants, diluents (also known as bulking agents or fillers), binders, lubricants, surface active agents, disintegrants and the like. The term also includes agents such as colouring agents, preserving agents and coating agents. Such excipients will generally be present in admixture within the tablet.

Examples of processing aids include, but are not limited to, microcrystalline cellulose and silicified microcrystalline cellulose.

Examples of solubilisers include, but are not limited to, ionic surfactants (including both ionic and non-ionic surfactants) such as sodium lauryl sulphate, cetyltrimethylammonium bromide, polysorbates (such as polysorbate 20 or 80), poloxamers (such as poloxamer 188 or 207), and macrogols.

Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet is generally between about 0.5-5% w/w. In one embodiment, the lubricant is sodium stearyl fumarate.

Examples of disintegrants include, but are not limited to, starches, celluloses, cross-linked PVP (crospovidone) (Type A or Type B), sodium starch glycolate (Type A or Type B), croscarmellose sodium, etc. In one embodiment, the disintegrant is sodium starch glycolate (Type A). In a further embodiment, the disintegrant is crospovidone (Type B).

Examples of diluents (also known as bulking agents or fillers) include, but are not limited to, starches, maltodextrins, polyols (such as lactose), and celluloses. For example, the diluent may be selected from mannitol, microcrystalline cellulose, silicified microcrystalline cellulose and lactose monohydrate. In one embodiment, the diluent is mannitol.

Examples of binders include, but are not limited to, cross-linked PVP, HPMC, sucrose, starches, etc. In one embodiment, the binder is a povidone. In a further embodiment, the binder is povidone K29/32.

In one embodiment, tablet formulations provided herein are uncoated. In a further embodiment, tablet formulations provided herein are coated. Although uncoated tablets may be used, it is more usual in the clinical setting to provide a coated tablet, in which case a conventional coating may be used.

Film coatings are known in the art. They can be composed of hydrophilic polymer materials and include, but are not limited to, polysaccharide materials, such as hydroxypropyl methylcellulose (HPMC), methylcellulose, hydroxyethyl cellulose (HEC), hydroxypropyl cellulose (HPC), poly(vinylalcohol-co-ethylene glycol) and other water soluble polymers. Though in one embodiment the water soluble material included in the film coating of the embodiments disclosed herein includes a single polymer material, in certain other embodiments it is formed using a mixture of more than one polymer. In one embodiment, the coating is yellow.

Suitable coatings include, but are not limited to, polymeric film coatings such as those comprising polyvinyl alcohol e.g. Opadry II yellow film coat. The amount of coating is generally from about 1% to about 5% w/w of the tablet. In one embodiment, the coating is about 4.5% w/w of the tablet.

In a second aspect, the present invention provides a process for making a dispersible tablet formulation comprising dolutegravir or a pharmaceutically acceptable salt thereof, abacavir or a pharmaceutically acceptable salt thereof, lamivudine and at least one taste masking agent comprising mixing dolutegravir or a pharmaceutically acceptable salt thereof, abacavir or a pharmaceutically acceptable salt thereof, lamivudine and at least one taste masking agent.

In one embodiment, granules of dolutegravir or a pharmaceutically acceptable salt thereof are first separately prepared prior to blending with extragranular components including, for example, lamivudine and at least one taste masking agent.

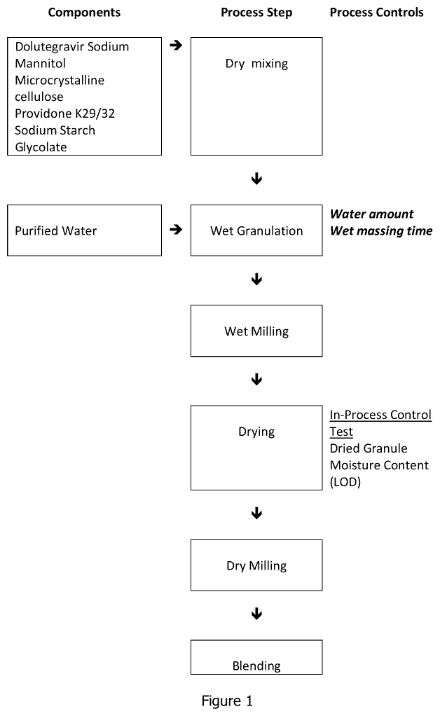

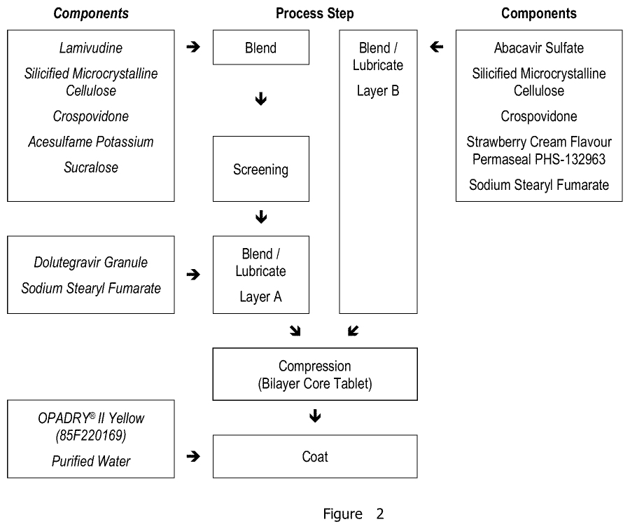

To make the dolutegravir granules, the active is first mixed with one or more of the aforementioned excipients in a suitable blender to blend the materials. In one embodiment, dolutegravir (as dolutegravir sodium) is admixed with a first amount of excipients by high shear granulation. This mixture is wet granulated and wet milled and the granules are then dried and dry milled. Thereafter, a second amount of excipients are added to the granules and further blended. The final dolutegravir granules are collected in a suitable container. Flow Diagrams for dolutegravir sodium granule manufacture and subsequent blending to form a multilayer tablet are shown in FIGS. 1 and 2.

In one embodiment, the dolutegravir sodium granules are blended with lamivudine, a processing aid, a disintegrant, two sweeteners and a lubricant to form the first layer of a bilayer tablet formulation. In another embodiment, the dolutegravir sodium granules are blended with lamivudine, silicified microcrystalline cellulose, crospovidone, acesulfame potassium, sucralose and sodium stearyl fumarate to form the first layer of a bilayer tablet formulation. In order to form the second layer of the bilayer tablet formulation, in one embodiment abacavir hemisulfate is blended with a processing aid, a disintegrant, a flavouring agent and a lubricant. In another embodiment abacavir hemisulfate is blended with silicified microcrystalline cellulose, crospovidone, strawberry cream flavour and sodium stearyl fumarate.

The first layer and second layer may be compressed separately and subsequently combined. However, more typically, the first layer is formed by compression and subsequently the second layer is compressed directly onto the first layer. In one embodiment, the choice of layer order in the tableting of multilayer tablets may have an impact on the properties of the tablets (e.g. the adhesion of the layers within the tablet).

In one embodiment, the methods will include a step of coating the tablet cores after compression, e.g. with a film coating as described above.

Tabletting methods are well known in the art of pharmacy. Techniques and formulations generally are found in Remington's Pharmaceutical Sciences (Mack Publishing Co., Easton, Pa.), which is hereby incorporated by reference herein in its entirety. A tablet can be made by compression or moulding. Compressed tablets may be prepared by compressing in a suitable machine the active ingredient and one or more excipients in a free-flowing form such as a powder or granules.

In another embodiment, the compressed tablet is film coated.

In one embodiment, the dispersible tablets of the invention are are coated and weigh about 397 mg each.

In a third aspect, the present invention provides a dispersible tablet formulation comprising dolutegravir or a pharmaceutically acceptable salt thereof, abacavir or a pharmaceutically acceptable salt thereof, lamivudine and at least one taste masking agent for use in therapy.

In a fourth aspect, the present invention provides a dispersible tablet formulation comprising dolutegravir or a pharmaceutically acceptable salt thereof, abacavir or a pharmaceutically acceptable salt thereof, lamivudine and at least one taste masking agent for use in the treatment of HIV infection, in particular HIV infection in a pediatric patient.

In a fifth aspect, the present invention provides a method of treating HIV infection, for example HIV infection in a pediatric patient, which method comprises administering to said patient a dispersible tablet formulation comprising dolutegravir or a pharmaceutically acceptable salt thereof, abacavir or a pharmaceutically acceptable salt thereof, lamivudine and at least one taste masking agent.

The dispersible tablet formulations of the invention are typically administered once per day.

The dispersible tablet formulations of the invention are typically administered indefinitely to maintain the desired therapeutic effect. It will be further understood by such skilled artisans that suitable dosing regimens may require adjustment for an individual patient.

The dispersible tablet formulations of the invention typically comprise 5 mg of dolutegravir in free acid form, 60 mg of abacavir in free based form and 30 mg of lamivudine. As the skilled person will appreciate, the number of tablets required per dose may depend on the age and size of the patient.

In a sixth aspect, the present invention provides a kit comprising a dispersible tablet formulation comprising dolutegravir or a pharmaceutically acceptable salt thereof, abacavir or a pharmaceutically acceptable salt thereof, lamivudine and at least one taste masking agent, together with instructions for the use thereof for the treatment of HIV infection.

In one embodiment, the kit comprises 90 tablets, for example 90 tablets in a 60 cc bottle with a child-resistant closure. In another embodiment, the kit comprises a dosing cup, for example a 60 ml dosing cup, and syringe, for example a 25 ml. In a further embodiment, the kit comprises instructions regarding the amount of water in which to disperse the tablet(s). For example, for a dose of 3 tablets, the tablets are typically dispersed in about 15 ml of water. For a dose of from 4 to 6 tablets, the tablets are typically dispersed in about 20 ml of water. An additional amount of water, for example about 15 ml, may be used to rinse the dosing cup.

In a seventh aspect, the present invention provides a combination of a dispersible tablet formulation comprising dolutegravir or a pharmaceutically acceptable salt thereof, abacavir or a pharmaceutically acceptable salt thereof, lamivudine and at least one taste masking agent, together with one or more other therapeutic agents.

In one embodiment, the other therapeutic agent is a nucleoside reverse transcriptase inhibitor such as didanosine, emtricitabine, stavudine, tenorfovir alafenamide fumarate, tenofovir disoproxil fumarate or zidovudine; a non-nucleoside reverse transcriptase inhibitor such as delaviridine, doravine, efavirenz, etravirine, nevirapine or rilpivirine; a protease inhibitor such as atazanavir, darunavir, fosamprenavir, indinavir, lopinavir, nelfinavir, saquinavir or tipranavir; a fusion inhibitor such as enfuvirtide; a CCRS antagonist such as maraviroc; a cytochrome P4503A inhibitor such as ritonavir or cobicistat; an integrase inhibitor such as dolutegravir, raltegravir, elvitegravir, bictegravir or cabotegravir; or a post attachment-inhibitor such as ibalizumab-uiyk.

The individual components of such combinations may be administered either sequentially or simultaneously in separate or combined formulations.

### EXAMPLES

### Triumeq Dispersible Bilayer Tablet

TABLE 1

| | | | |
|---|---|---|---|
| | Quantity per tablet | | |
| | (mg) [and potential | | |
| Ingredients | range for each quantity] | Function | Rationale |

Doutegravir/Lamivudine
layer (180 mg)
Dolutegravir Granule
(28.8 mg)

| API (dolutegravir sodium) | 5.26 | [4.5-6.6] | Active | Granule used in Tivicay |
|---|---|---|---|---|
| D-Mannitol | 14.54 | [12.4-18.2] | Intermediate | and Triumeq products |
| Microcrystalline Cellulose | 6 | [6.9-7.2] | | |
| Povidone | 1.5 | [1.3-1.9] | | |
| Sodium Starch Glycolate | 1.5 | [1.3-1.9] | | |
| Purified Water | qs | | | |
| Extra granular | | | | |
| API (lamivudine) | 30 | [25.5-37.5] | Active | |
| | | | ingredient | |
| Silicified Microcrystalline | 86.4 | [73.4-108] | Compression | Compression and flow |
| Cellulose | | | aid | enhancer, good |
| | | | | mouthfeel |
| Crospovidone | 14.4 | [12.2-18] | Disintegrant | Quick dispersion |
| Acesulfame Potassium | 12 | [10.2-15] | Taste | Sweetener to mask API |
| | | | masker | bitterness in the |
| | | | | dispersion |
| Sucralose Powder | 4.8 | [4.1-6] | Taste | Sweetener to mask API |
| | | | masker | bitterness in the |
| | | | | dispersion |
| Sodium Stearyl Furmarate | 3.6 | [3.1-4.5] | Lubricant | Tablet ejection |
| Abacavir LAYER (200 mg) | | | | |
| Abacavir hemi-sulfate, API | 70.2 | [59.7-87.8] | Active | |
| | | | ingredient | |
| Silicified Microcrystalline | 108 | [91.8-135] | Compression | Compression and flow |
| Cellulose | | | aid | enhancer, good |
| | | | | mouthfeel |
| Crospovidone | 14 | [11.9-17.5] | Disintegrant | Disintegrant, required |
| | | | | for quick dispersion |
| Strawberry Cream Flavour | 3.8 | [3.3-4.8] | Taste | Flavour, to improve |
| | | | masker | dispersion perception |
| Sodium Stearyl Furmarate | 4 | [3.4-5] | Lubricant | Tablet ejection |
| FILM COAT | | | | |
| Opadry II, yellow | 17.1 | [14.5-21.4] | Coat | Used at 12% solids. |
| | | | | PVA coat with reduced |
| | | | | level of titanium dioxide |
| | | | | (to minimise scuffing |
| | | | | potential). Coat protects |
| | | | | against Abacavir (skin |
| | | | | sensitizer) and aids in |
| | | | | swallowing. |

### Specification for Triumeq Dispersible Bilayer Tablet

TABLE 2

| Test | Acceptance Criteria | Results |
|---|---|---|
| Description | Capsule shaped, biconvex, yellow, | Conforms |
| | film coated tablet, debossed with | |
| | ‘SV WTU’ on one face | |
| Identification by HPLC | | |
| Dolutegravir | The retention time of the peak due | Conforms |
| | to dolutegravir in the sample | |
| | chromatogram is concordant with | |
| | that of the dolutegravir peak in the | |
| | standard reference chromatogram | |
| Abacavir | The retention time of the peak due | Conforms |
| | to abacavir in the sample | |
| | chromatogram is concordant with | |
| | that of the abacavir peak in the | |
| | standard reference chromatogram | |
| Lamivudine | The retention time of the peak due | Conforms |
| | to lamivudine in the sample | |
| | chromatogram is concordant with | |
| | that of the lamivudine peak in the | |
| | standard reference chromatogram | |
| Mean Content by HPLC | | |
| (% label claim) | | |
| Dolutegravir | 90.0-110.0 | 103.9 |
| Abacavir | 90.0-110.0 | 101.9 |
| Lamivudine | 90.0-110.0 | 101.4 |
| Dolutegravir Drug-related | | |
| Impurities Content (% area) | | |
| Any dolutegravir related | Not greater than 0.2 | <0.05 |
| unspecified impurity | | |
| Total dolutegravir related | Not greater than 1.0 | 0.05 |
| impurities | | |
| Abacavir and Lamivudine | | |
| Drug-related Impurities | | |
| Content (% area) | | |
| Any abacavir related | Not greater than 0.2 | <0.05 |
| unspecified impurity | | |
| Total abacavir related | Not greater than 1.0 | 0.11 |
| impurities | | |
| Any lamivudine related | Not greater than 0.2 | <0.05 |
| unspecified impurity | | |
| Total lamivudine related | Not greater than 0.6 | 0.24 |
| impurities | | |
| Disintegration Time | Within 3 minutes | 1 minute |
| Dissolution (% released) | | Stage 1 Conforms |
| Dolutegravir | Complies with the requirements of | 91 (87-95) |
| | USP <711> and Ph. Eur. 2.9.3, | |
| | where Q = 75% at 60 minutes | |
| Abacavir | Complies with the requirements of | 101 (99-104) |
| | USP <711> and Ph. Eur. 2.9.3, | |
| | where Q = 75% at 60 minutes | |
| Lamivudine | Complies with the requirements of | 99 (92-106) |
| | USP <711> and Ph. Eur. 2.9.3, | |
| | where Q = 75% at 60 minutes | |
| Uniformity of Dosage Units | | Stage 1 Conforms |
| Dolutegravir | Complies with the requirements of | AV = 4.9 |
| | USP <905> and Ph. Eur. 2.9.40 | |
| Abacavir | Complies with the requirements of | AV = 3.2 |
| | USP <905> and Ph. Eur. 2.9.40 | |
| Lamivudine | Complies with the requirements of | AV = 8.7 |
| | USP <905> and Ph. Eur. 2.9.40 | |
| Fineness of Dispersion | A smooth dispersion is produced | Conforms |
| | which passes through a sieve | |
| | screen with a nominal mesh | |
| | aperture of 710 μm | |

### Pharmacokinetics

### Relative Bioavailability

TABLE 3

| Treatment | | |
|---|---|---|
| (TRT) | Product | Dispersion Time |
| A | Reference (Adult TRIUMEQ ® tablet) | Direct to mouth |
| | 50 mg DTG + 600 mg ABC + 300 mg 3TC | |
| B | Trii Dispersible tablet (dispersed in purified H₂O) | Dispersion (within 30 min) |
| | 5 mg DTG + 60 mg ABC + 30 mg 3TC | |
| C | Trii Dispersible tablet | Direct to mouth (DM) |
| | 5 mg DTG + 60 mg ABC + 30 mg 3TC | |

### Results:

TABLE 4

| | | | |
|---|---|---|---|
| | AUC_{0-inf} | AUC₀₋ₜ | Cₘₐₓ |
| Treatment | GMR(90% CI) | GMR(90% CI) | GMR(90% CI) |

Dolutegravir

| | | | |
|---|---|---|---|
| Dispersion/Tri adult tab B/A | 1.69 (1.56, 1.84) | 1.70 (1.57, 1.84) | 1.74 (1.60, 1.89) |
| DM/Tri adult tab C/A | 1.35 (1.25, 1.47) | 1.35 (1.25, 1.47) | 1.36 (1.25, 1.48) |
| Dispersion/DM B/C | 1.25 (1.16, 1.36) | 1.26 (1.16, 1.36) | 1.28 (1.17, 1.39) |

Abacavir

| | | | |
|---|---|---|---|
| Dispersion/Tri adult tab B/A | 1.04 (1.01, 1.07) | 1.04 (1.01, 1.07) | 1.05 (0.99, 1.12) |
| DM/Tri adult tab C/A | 1.02 (0.99, 1.05) | 1.02 (0.99, 1.05) | 0.98 (0.92, 1.04) |
| Dispersion/DM B/C | 1.02 (0.99, 1.05) | 1.02 (0.99, 1.05) | 1.08 (1.01, 1.15) |

Lamivudine

| | | | |
|---|---|---|---|
| Dispersion/Tri adult tab B/A | 1.00 (0.96, 1.05) | 1.00 (0.95, 1.05) | 0.94 (0.87, 1.01) |
| DM/Tri adult tab C/A | 0.95 (0.90, 0.99) | 0.94 (0.90, 0.99) | 0.91 (0.84, 0.98) |
| Dispersion/DM B/C | 1.06 (1.01, 1.11) | 1.06 (1.01, 1.11) | 1.03 (0.96, 1.11) |

Dolutegravir ˜1.6× exposure when dosed as dispersion vs adult immediate release tablet (similar result for Tivicay 5 mg dispersible)
Dolutegravir ˜1.25× exposure when dosed direct to mouth vs adult immediate release tablet (lower exposure than seen with Tivicay 5 mg where dispersed and direct to mouth were the same).
No differences in exposure for Lamivudine or Abacavir when dispersed or direct to mouth vs. the IR tablet

### Palatability Data

Palatability data was also collected with this study, where 88% of subjects (15 of 17) rated the palatability as ‘Neutral/Acceptable’; 11% (2 of 17) ‘Unacceptable’.

### Initial Relative Bioavailability PK Study

This showed that the level of DTG solubility does not impact the overall DTG levels in plasma.

### Treatments:

Treatment (TRT) A: Reference 4×10 mg DTG tablet+Kivexa (ABC 600 mg/3TC 300 mg)

Treatment B-E: Triumeq bilayer tablets—4 tablets (10 mg DTG/ABC 150 mg/3TC 75 mg) dispersed in high mineral content water (Contrex)* or purified water. This formulation did not contain flavour or sweetener, so these were added extemporaneously.

*Contrex water contains a very high level of minerals including calcium, purified water is mineral free.

TABLE 5

| | | Dispersion | | Estimate of DTG |
|---|---|---|---|---|
| Treatment | Product | time | Administration | solubility |
| A | CONTROL 4 × 10 mg | Direct to | With water | N/A |
| | tablet + Kivexa | mouth | | |
| B | Bi-layer tablet | Immediate | Contrex water (Ca++) | Approx. 45% DTG |
| C | Bi-layer tablet | 30 mins | plus flavour and sweetener | Approx. 45% DTG |
| D | Bi-layer tablet | Immediate | Purified water plus | Approx. 70% DTG |
| E | Bi-layer tablet | 30 mins | flavour and sweetener | Approx. 16% DTG |

Abacavir and lamivudine are fully soluble when dispersed and showed no differences in exposure across all 5 arms.

Dolutegravir showed ˜1.6× higher exposure compared to the reference with no impact on PK relating to solubility across the range studied (16-70%).

TABLE 6

| Parameter (units) | Test vs Reference | n | Ratio | CI (90%) |
|---|---|---|---|---|
| AUC_{0-inf} (h*ug/mL) | B vs A | 20 | 1.56 | (1.49, 1.64) |
| | C vs A | 20 | 1.53 | (1.46, 1.61) |
| | D vs A | 20 | 1.58 | (1.51, 1.65) |
| | E vs A | 19 | 1.54 | (1.47, 1.62) |
| Cmax (ug/mL) | B vs A | 20 | 1.59 | (1.50, 1.70) |
| | C vs A | 20 | 1.56 | (1.46, 1.66) |
| | D vs A | 20 | 1.56 | (1.46, 1.66) |
| | E vs A | 19 | 1.58 | (1.48, 1.68) |

1. A dispersible tablet formulation comprising dolutegravir or a pharmaceutically acceptable salt thereof, abacavir or a pharmaceutically acceptable salt thereof, lamivudine and at least one taste masking agent.

2. A dispersible tablet formulation according to claim 1 which is a multilayer dispersible tablet formulation.

3. A dispersible tablet formulation according to claim 2 which is a bilayer dispersible tablet formulation.

4. A dispersible tablet formulation according to claim 2, wherein the dolutegravir or a pharmaceutically acceptable salt thereof is present within a separate layer to the abacavir or a pharmaceutically acceptable salt thereof

5. A dispersible tablet formulation according to claim 2, wherein the dolutegravir or a pharmaceutically acceptable salt thereof and lamivudine are present within a separate layer to the abacavir or a pharmaceutically acceptable salt thereof

6. A dispersible tablet formulation according to claim 1, wherein the at least one taste masking agent comprises at least one sweetener.

7. A dispersible tablet formulation according to claim 6 comprising two sweeteners.

8. A dispersible tablet formulation according to claim 7 wherein the sweeteners are acesulfame potassium and sucralose.

9. A dispersible tablet formulation according to claim 1, wherein the at least one taste masking agent comprises two sweeteners and a flavouring agent.

10. A dispersible tablet formulation according to claim 1, wherein the tablet comprises a coating.

11. A process for making a dispersible tablet formulation as claimed in claim 1 comprising mixing dolutegravir or a pharmaceutically acceptable salt thereof, abacavir or a pharmaceutically acceptable salt thereof, lamivudine and at least one taste masking agent.

12. A dispersible tablet formulation as claimed in claim 1 for use in therapy.

13. A dispersible tablet formulation as claimed in claim 1 for use in the treatment of HIV infection.

14. A method of treating HIV infection, which method comprises administering to said patient a dispersible tablet formulation as claimed in claim 1.
